(19) United States
(12) Patent Application Publication
(10) Pub. No.: US 2010/0144898 A1
(43) Pub. Date: Jun. 10, 2010

(54) MIXTURE COMPRISING AN ALKYLPOLYGLUCOSIDE, A COSURFACTANT AND A POLYMER ADDITIVE

(76) Inventors: Joerg Adams, Bad Neuenahr (DE); Juergen Allgaier, Aachen (DE); Christian Frank, Horrem (DE)

Correspondence Address: JACOBSON HOLMAN PLLC, 400 SEVENTH STREET N.W., SUITE 600, WASHINGTON, DC, 20004, US

(21) Appl. No.: 12/451,082

(22) PCT Filed: Apr. 28, 2008
(86) PCT No.: PCT/EP2008/055176

§ 371 Date: Feb. 4, 2010

(30) Foreign Application Priority Data
Apr. 27, 2007 (DE) 10 2007 020 426.6
Jul. 26, 2007 (DE) 10 2007 035 388.1

(51) Int. Cl. A61K 8/84; A61Q 90/00; A23D 7/00; C11D 17/00

(52) U.S. Cl. 514/772.1; 426/602; 510/417

(57) ABSTRACT

A mixture comprising a component I comprising 80-20% by weight of a first surfactant component I₁, which is an alkylpolyglucoside containing 1-2 glucoside moieties and a hydrocarbon moiety, especially an alkyl residue of 6-16 carbon atoms, 20-80% by weight of a component I₂, which is a second surfactant containing alcohol groups other than an alkylpolyglucoside, the weight proportions being based on component I only; and

a polymeric additive as component II, wherein the polymeric additive, as component II_{I}, contains at least one water-soluble moiety and at least one hydrophobic moiety, the ratio of the number average molecular weights of all water-soluble moieties to the number average molecular weights of all hydrophobic moieties being from 2:1 to 1000:1 or from 3.1 to 1000:1, especially from 5:1 to 200:1, in particular from 10:1 to 50:1, wherein each of said at least one hydrophobic moieties has a number average molecular weight of at most 1000 g/mol; or the polymeric additive, as component II₂, contains at least one water-soluble moiety and at least one hydrophobic moiety and is an amphiphilic comb polymer including a backbone with two or more side chains attached to said backbone, wherein the side chains are distinguished from one another and/or from the backbone in terms of their amphiphilic character; or the polymeric additive, as component II₃, contains at least one water-soluble moiety and at least one hydrophobic moiety wherein said polymeric additive as component II₃ is an AB diblock copolymer or an ABA or BAB triblock copolymer with water-soluble A blocks and hydrophobic B blocks.

The invention relates to a mixture comprising two components I and II, to an emulsion that can be prepared from said mixture and may also be in the form of a microemulsion, thus especially a bicontinuous microemulsion, and to a cleaning agent, a cosmetic article and a food that comprise said emulsion, and to the use of the cleaning agent.

Surfactants are detergent substances contained in laundry detergents, dishwashing detergents and shampoos. They have a characteristic structure and include at least one hydrophilic and one hydrophobic moiety. They have an amphiphilic character. If the stabilizing effect on water-oil mixtures is the important characteristic, then these amphiphilic substances are employed'as emulsifiers.

Surfactants reduce the interfacial tension between immiscible phases a hydrophilic (water-soluble, lipophobic), mostly aqueous, phase and a hydrophobic (oil-soluble, lipophilic) phase. Such liquid two-phase mixture are referred to as “emulsions”.

Conventional emulsions may contain hydrophilic and hydrophobic phases in different volume proportions. They include a continuous phase and a disperse phase which is contained in the continuous phase in the form of very small spheres stabilized by surfactants occupying their surface. Depending on the nature of the continuous phase, the emulsions are referred to as “oil-in-water” or “water-in-oil”.

A fundamental distinction is made between emulsions and microemulsions. While microemulsions are thermodynamically stable, emulsions will segregate due to their instability. On a microscopic scale, this difference is manifested in the fact that the emulsified liquids in microemulsions are contained in smaller liquid volumes (e.g., 10⁻¹⁵ μl) as compared to emulsions (e.g., 10⁻¹² μl), as described in DE 10 2005 049 765 A1. Thus, thermodynamically unstable emulsions have larger structures.

In microemulsions, lamellar mesophases may occur. Lamellar mesophases result in optical anisotropy and increased viscosity. Such properties are undesirable for cleaning agents, for example. In addition, phase separation often occurs when lamellar phases coexist with microemulsions.

Microemulsions consist of at least three components, namely oil, water and a surfactant [1-7]. Oil and water are not miscible and therefore form domains on a nanoscale. The surfactant mediates between these two components and allows for a macroscopically homogeneous mixture. On a microscopic scale, the surfactant forms a film between the oil and water domains. Microemulsions are macroscopically homogeneous have an optically isotropic behavior and, in contrast to emulsions, are thermodynamically stable. There are w/o and o/w droplet microemulsions, wherein water droplets are surrounded by oil or oil droplets are surrounded by water, respectively. About equal proportions of oil and water favor the formation of a bicontinuous microemulsion.

Characteristic of the efficiency of a surfactant is the minimum amount of surfactant required to stabilize emulsions over the desired period of time or maintain a microemulsion.

Microemulsions have been intensively studied in the field of fundamental science [8, 9]. The knowledge gained thereby is substantially based on the use of pure and defined components: deionized water, chemically pure oils and pure surfactants. With technical microemulsions, the components usually consist of mixtures of substances. This considerably changes the ratio of the phases, and the knowledge gained from simplified models in fundamental research cannot be transferred to technical applications so easily. Another difficulty resides in the low thermal stability of microemulsions, since practical formulations require stability over a broad range of temperatures. Especially systems based on the widely used fatty alcohol ethoxylates are stable only in a very narrow temperature window of a few 20 C., or extremely high surfactant concentrations must be used. In contrast, microemulsions prepared by means of sugar surfactants may be stable over broader temperature ranges. Similarly, mixtures of non-ionic and ionic surfactants may also be employed. In this case, the different thermal behavior of the non-ionic and ionic surfactants is utilized. However, sugar surfactants and mixtures of non-ionic surfactants also have drawbacks. Microemulsions made of sugar surfactants can only be prepared by using cosurfactants. According to the state of the art, monovalent alcohols, such as hexanol or octanol, are used as said cosurfactants. Microemulsions containing ionic surfactants are sensitive towards changes of the salt concentration.

Since the research on microemulsions takes place mainly in the field of fundamental research, it has been hardly taken care in this field that surfactants be used that include a low hazard potential or are prepared from renewable raw materials. For technical applications, this may be of great importance since surfactant contents of 20-30% are usual in conventional microemulsions in order to achieve a sufficiently broad temperature stability. In such concentrations, surfactants have a hazard potential that is no longer negligible. In particular, they have an irritant effect on the skin and eyes. An exception is this respect are alkylpolyglucosides, which are prepared from renewable raw materials and have a moderate hazard potential and are moreover relatively skin-friendly. In contrast, sorbitan esters, which have a very low hazard potential and are also essentially prepared from renewable raw materials, have hardly been studied to date in terms of their use in microemulsions.

DE-A-198 39 054 discloses a process for enhancing the efficiency of surfactants while simultaneously suppressing lamellar mesophases, a process for the stabilization of the temperature situation of the one-phase region for mixtures of oil, water and surfactant, a process for increasing the structural size of emulsified liquid particles in microemulsions, and a process for reducing the interfacial tension of oil-water mixtures in which AB block copolymers having a water-soluble block A and a water-insoluble block B are added. The polymers consist of a water-soluble block A and a hydrophobic block. The lower limits of the number average molecular weights for A and B are around 500 g/mol. This process is suitable for the preparation of microemulsions.

DE-A-103 23 180 describes mixtures containing a surfactant and a cosurfactant, characterized in that an amphiphilic comb polymer having a backbone with two or more side chains attached to said backbone is employed as the cosurfactant, wherein the side chains are distinguished from one another and/or from the backbone in terms of their amphiphilic character. The cosurfactant is suitable for enhancing the efficiency in microemulsions.

Further, DE-A-44 17 476 discloses a microemulsion containing alkylglycosides and fatty acid polyol partial esters. The microemulsion is to exist in a broad range; however, a temperature range in which the microemulsion is stable is not disclosed.

DE-A-198 24 236 proposes a process for cleaning printing machines or printing formes in which the contaminants are removed from the surfaces to be cleaned by washing with a microemulsion containing water, a surface-active agent and an organic solvent immiscible with water.

U.S. Pat. No. 5,719,113 discloses cleaning agents comprising an antibacterial substance, a non-ionic surfactant and an amphoteric surfactant. In contrast to the mixture according to the invention, it does not disclose a second surfactant containing alcohol groups.

The object of the invention is to provide a mixture having improved properties that can be processed into an emulsion, especially a microemulsion.

This emulsion, especially microemulsion, is to require a lower amount of surfactants and be stable in a broader temperature range. In one embodiment, the emulsion, especially microemulsion, according to the invention has the advantage of being free or almost free from volatile organic compounds (VOCs). According to the 31st Regulation for Implementing the Federal Immission Control Act (31. BimschV), Section 2, No. 11, a “VOC” is defined as a volatile organic compound having a vapor pressure of 0.01 kPa or more at 293,15 Kelvin. The VOC include, for example, compounds from the groups of substances of alkanes/alkenes, aromatics, terpenes, halohydrocarbons, esters, aldehydes and ketones.

The above object is achieved by a mixture according to the invention as defined in claim 1.

The mixture according to the invention includes a component I comprising 80-20% by weight of a first surfactant component I₁, which is an alkylpolyglucoside containing 1-2 glucoside moieties and a hydrocarbon moiety, especially an alkyl residue of 6-16 carbon atoms, 20-80% by weight of a component I₂, which is a second surfactant containing alcohol groups other than an alkylpolyglucoside, the weight proportions being based on component I only; and

a polymeric additive as component II, wherein the polymeric additive, as component II₁, contains at least one water-soluble moiety and at least one hydrophobic moiety, the ratio of the number average molecular weights of all water-soluble moieties to the number average molecular weights of all hydrophobic moieties being from 2:1 to 1000:1 or from 3.1 to 1000:1, especially from 5:1 to 200:1, in particular from 10:1 to 50:1, wherein each of said at least one hydrophobic moieties has a number average molecular weight of at most 1000 g/mol; or the polymeric additive, as component II₂, contains at least one water-soluble moiety and at least one hydrophobic moiety and is an amphiphilic comb polymer including a backbone with two or more side chains attached to said backbone, wherein the side chains are distinguished from one another and/or from the backbone in terms of their amphiphilic character; or the polymeric additive, as component II₃, contains at least one water-soluble moiety and at least one hydrophobic moiety wherein said polymeric additive as component II₃ is an AB diblock copolymer or an ABA or BAB triblock copolymer with water-soluble A blocks and hydrophobic B blocks.

The polymeric additives of components II₁, II₂ or II₃ may also be in combination in said mixture.

Said component II, which is contained as a polymeric additive in the mixture according to claim 1, seems to result in an increased efficiency of the surfactants in component I.

In addition to reasons of cost, saving surfactant is advantageous also for ecological or health reasons. Surfactants are substances of particular ecological relevance whose environmental compatibility must be ensured.

Another advantage of the saving of surfactants is seen when surfactants have disturbing effects in the application of the microemulsion. For example, cosmetics may be mentioned whose surfactant content should be as low as possible due to the skin-affecting effect that may occur with sensitive skin, or a possibly occurring eye-irritant effect of the surfactants. The same applies especially to foods. The load on the consumer from surfactants should be as low as possible. The present invention contributes to this.

In one embodiment, the emulsion according to the invention resulted in a lower time expenditure for cleaning as compared to the prior art.

The mixture according to the invention includes components I and II. Component I in turn comprises from 80 to 20% by weight of component I_{I}, which is an alkyl-polyglucoside containing 1-2 glucoside moieties and a hydrocarbon moiety, especially an alkyl residue of 6-16 carbon atoms, and further from 20 to 80% by weight of component I₂, which is a cosurfactant containing alcohol groups other than an alkylpolyglucoside. The above weight proportions are based on component I only.

Consequently, according to the invention, component I₂ is not propylene glycol.

In one embodiment of the mixture according to the invention, component I₂ has an HLB value of 1-11 or 3-11 or 5-11 or 1-5 or 3-5 in aqueous solution. The HLB value describes the hydrophilic and lipophilic proportions of a surfactant.

According to Griffin, the HLB value is calculated as follows [10]:

HLB=20·Mₕ/M

where Mₕ=molecular weight of the hydrophilic portion of a molecule; M=molecular weight of the whole molecule.

According to the invention, surfactants of component I₂ that are skin-friendly are employed, in particular. Examples thereof include sorbitan esters. In addition, other surfactants (emulsifiers) admissible under food law may also be employed.

In one embodiment of the mixture according to the invention, component Iᵢ is more hydrophilic than component I₂. This means that the HLB value of component I_{I} is higher than that of component I₂.

For example, the mixture according to the invention may be prepared in such a way that component I₁ has an HLB value of 11-19, especially 11-15, and component I₂ has an HLB value of 1-11, especially 3-11 or 5-11 or 1-5 or 3-5.

According to claim 1, component II according to the invention is a polymeric additive that includes either component II₁ or II₂ or II₃. Further, component II₁ comprises at least one water-soluble moiety and at least one hydrophobic moiety, the ratio of the number average molecular weights of all water-soluble moieties to the number average molecular weights of all hydrophobic moieties being from 2:1 to 1000:1, especially from 5:1 to 200:1, in particular from 10:1 to 50:1, wherein each of said at least one hydrophobic moieties has a number average molecular weight of at most 1000 g/mol.

Component II₂ also contains at least one water-soluble moiety and at least one hydrophobic moiety. It is an amphiphilic comb polymer including a backbone with two or more side chains attached to said backbone, wherein the side chains are distinguished from one another and/or from the backbone in terms of their amphiphilic character.

Component II₃ contains at least one water-soluble moiety and at least one hydrophobic moiety, being an AB diblock copolymer or an ABA or BAB triblock copolymer with water-soluble A blocks and hydrophobic B blocks.

In another embodiment, the mixture according to the invention comprises 80-99% by weight, especially 85-95% by weight, of component I and 1-20% by weight, especially 5-15% by weight, of component II.

The invention also relates to an emulsion obtainable by diluting the mixture according to the invention with an aqueous solution and an oily phase. This results in the formation of an emulsion of the hydrophilic and hydrophobic phases, which is stabilized by the mixture according to the invention.

In one embodiment, said emulsion is characterized by being a microemulsion, in particular being a bicontinuous microemulsion. Bicontinuous microemulsions comprise two phases, a hydrophobic and a hydrophilic phase, in the form of extended coexisting and intertwined domains at whose interface stabilizing surface-active agents are enriched in a monomolecular layer (cf. [11]). Microemulsions form very readily and spontaneously because of the very low interfacial tension when the individual components water, oil and a suitable surface-active system are mixed together. Since the domains have very small sizes on the order of a few nanometers in at least one dimension, microemulsions often appear visually transparent and are thermodynamically stable, i.e., without a time limit, in a particular range of temperatures, depending on the surface-active system employed. If microemulsions have low surfactant contents, they may also be turbid.

In another embodiment, the microemulsion according to the invention may be a w/o or o/w droplet microemulsion, wherein water droplets are surrounded by oil or oil droplets are surrounded by water, respectively.

The appropriate mass ratio of oily phase to aqueous phase strongly depends on the field of application and may be optimized by the skilled person in routine experiments. Thus, for example, a ratio of 0.01 may yield satisfactory results in the plant protection field, and a ratio of 0.7 in the field of household cleaning agents.

In another embodiment of the microemulsion according to the invention, the mass ratio of oily phase to aqueous phase is from 0.5 to 1.6. Such ratios are appropriate for industrial cleaning agents.

In another embodiment of the cleaning agent according to the invention, the mass ratio of oily phase to aqueous phase of the microemulsion is from 1.0 to 1.4.

In one embodiment, the oily phase of the emulsion includes mineral oils, especially aliphatic naphthenic hydrocarbons, such as petroleum spirit. This also includes dearomatized petroleum blends with 11-14 carbon atoms, dearomatized white spirits with 9-12 carbon atoms, specific dearomatized fractions with 9-10 carbon atoms as well as polar solvents, such as derivatives of carbonic acid (e.g., 4-methyl-1,3-dioxolan-2-one), derivatives of lactic acid, such as ethyl lactate, n-propyl lactate and 2-ethylhexyl lactate, and of dicarboxylic acids, such as dimethyl esters or diisobutyl esters of glutaric acid, adipic acids or succinic acid, as well as glycol ethers based on ethylene glycol and propylene glycol units, such as diethylene glycol monobutyl ether or dipropylene glycol dimethyl ether. The oily phase of the emulsion may further include triglycerides and products from the esterification and transesterification of vegetable oils, such as fatty acid methyl ester (e.g., rapeseed oil methyl ester or coco ester). These substances may also display surfactant activities.

Especially if triglycerides derived, for example, from plants or heavy hydrocarbon oils, especially aliphatic hydrocarbons, are used, an almost odorless emulsion can be prepared.

In another embodiment, the microemulsion according to the invention has no lamellar phase.

In one embodiment, the emulsion according to the invention comprises 80-99% by weight, especially 85-95% by weight, of component I, based on the total active surfactant content of the emulsion. Component I in turn comprises two components: component I₁ and component I₂ of the mixture according to the invention. In addition, the emulsion comprises 1-20% by weight, especially 5-15% by weight, of component II, based on the total active surfactant content of the emulsion, which is a polymeric additive as in the mixture according to the invention.

In one embodiment, the amount of the mixture according to the invention, based on the total amount of emulsion according to the invention, is 1-20%, especially 3-15% and in particular 3-10%.

In an additional embodiment, the emulsion includes further surfactants.

In one embodiment, the amphiphilic comb polymer (component II₂) is characterized in that the skeleton of the comb polymer is hydrophobic and all the side chains of the comb polymer are hydrophilic.

In another embodiment, the amphiphilic comb polymer is characterized by having repeating moieties [A]ₙ, [A′]ₘ and [X]ᵢ, wherein the moieties [A]ₙ and [A′]ₘ form the skeleton and the moiety [A′]ₘ has an anchoring function to bind the moieties [X]ᵢ forming the side chains, and wherein the variables n, m and i are molar fractions with

n+m+i=1;

n≧m; and

1>m.

In one embodiment of the invention, component I₂ comprises hydrocarbyl residues, especially 1-2 alkyl residues, preferably 1 to 1.5 alkyl residues, each having 8-20 carbon atoms, and a hydrophilic residue bearing more than one, but a maximum of 5, OH groups.

In one embodiment according to the invention, the alkylpolyglucoside of component I₁ has 1-1.5 glucoside moieties and a hydrocarbyl residue, especially an alkyl residue with 8-14 carbon atoms.

Another embodiment of the invention is characterized in that the hydrocarbyl residues, especially the alkyl residues, of component I₂ are connected with the hydrophilic residue via ether or ester groups.

In another embodiment, the hydrocarbyl residues, especially the alkyl residues, of component I₂ are connected with the hydrophilic residue via carbon bonds.

In one embodiment, the OH groups of component I₂ are ethoxylated. However, there are not more than 5, preferably not more than 2, ethylene oxide moieties per OH group.

In another embodiment, component I₂ comprises a hydrocarbyl residue, especially an alkyl residue with 10-18 carbon atoms, preferably 10-14 carbon atoms.

In still another embodiment, the hydrophilic residue of component I₂ comprises 1.5-3 OH groups.

In an additional embodiment, the hydrophilic residue of component I₂ is not ethoxylated.

In one embodiment, component I₂ is a sorbitan ester, such as sorbitan monolaurate or sorbitan monopalmitate, polysorbate, such as polysorbate 61 (POE(4)-sorbitan monostearate), glycerol monoester, a mixture of glycerol monoester and glycerol diester, a monoester or diester of pentaerythritol, a monoether or diether of pentaerythritol, 1,2-decanediol or 1,2-dodecanediol.

In another embodiment, said at least one hydrophobic moiety of component II₁ is provided at at least one chain end of a water-soluble moiety.

In an additional embodiment, said at least one hydrophobic moiety of component II₁ is a non-terminal substituent of a water-soluble moiety.

In a particular embodiment, said at least one hydrophobic moiety of component IIhd 1 is provided between at least two water-soluble moieties if more than at least one water-soluble moieties are present.

In one embodiment, the number average molecular weight of the water-soluble A blocks and the hydrophobic B blocks of the diblock copolymer or a triblock copolymer of component II₃ according to claim 1 is between 500 and 100,000 g/mol, especially between 2000 and 20,000 g/mol and especially between 3000 and 10,000 g/mol.

In one embodiment, the number average molecular weight of each hydrophobic moiety of component II₁ is between 80 and 1000 g/mol, especially between 110 and 500 g/mol, in particular between 110 and 280 g/mol.

In an additional embodiment, the number average molecular weight of each water-soluble moiety of component II₁ is at least 500 g/mol; the upper limit of the number average molecular weight depends on the field of application. Typically, the number average molecular weight is between 500 and 50,000 g/mol, especially between 900 and 20,000 g/mol, in particular between 2000 and 20,000 g/mol or between 3000 and 10,000 g/mol.

In another embodiment, the number average molecular weight of all the water-soluble moieties of component II is at least five times as high as the number average molecular weight of the hydrophilic fractions of component I.

In still another embodiment, the number average molecular weight of all the water-soluble moieties of component II is at least ten times as high as the number average molecular weight of the hydrophilic fractions of component I.

In one embodiment, the water-soluble moiety of component II comprises at least one of the molecules: polyethylene oxide, polyethylene glycol, copolymers of ethylene oxide and propylene oxide, polyacrolein, polyvinyl alcohol and its water-soluble derivatives, polyvinylpyrrolidone, polyvinylpyridine, polymethacrylic acid, polymaleic anhydride, polyformic acid, polyacrylic acid, polystyrenesulfonic acid and its water-soluble salts.

In an additional embodiment, the water-soluble moiety of component II₁ is a linear polymer.

One embodiment of the invention is characterized in that the water-soluble moiety of component II is not ionic in nature.

In another embodiment of the invention, the water-soluble moiety of component II may be ionic in nature.

In an additional embodiment, the water-soluble moiety of component II has at least two electric charges.

In another embodiment, the water-soluble moiety of component II is constituted of an ionic and a non-ionic component.

In one embodiment, the hydrophobic moiety of component II₁ is a hydrocarbyl residue, especially an alkyl residue.

In another embodiment, the hydrocarbyl residue, especially the alkyl residue, includes from 6 to 50 carbon atoms, preferably from 8 to 20 carbon atoms.

In one embodiment according to the invention, the hydrophobic moiety of component II is unsaturated.

In an additional embodiment, component II₁ is an alcohol ethoxylate consisting of a monovalent alcohol with 8-20 carbon atoms and 25-500 ethylene oxide moieties.

In one embodiment of the mixture according to the invention, the alkylpolyglucosides of component II have 1-1.5 glucoside moieties and a hydrocarbyl residue, especially an alkyl residue with 8-14 carbon atoms, or 1-2 glucoside moieties and a hydrocarbyl residue, especially an alkyl residue with 8-14 carbon atoms; component I₂ comprises hydrocarbyl residues, especially 1-2 alkyl residues, preferably 1 to 1.5 alkyl residues, each having 8-20 carbon atoms and a hydrophilic residue bearing more than one, but a maximum of 5, OH groups.

In an additional embodiment of the mixture according to the invention, the alkylpolyglucosides of component I₁ have 1-1.5 glucoside moieties and a hydrocarbyl residue, especially an alkyl residue with 8-14 carbon atoms, or 1-2 glucoside moieties and a hydrocarbyl residue, especially an alkyl residue with 8-14 carbon atoms; component I₂ is a sorbitan ester, a polysorbate, a glycerol monoester, a mixture of glycerol monoester and glycerol diester, a monoester or diester of pentaerythritol, a monoether or diether of pentaerythritol, 1,2-decanediol or 1,2-dodecanediol.

In another embodiment of the mixture according to the invention, the alkylpolyglucosides of component I₁ have 1-1.5 glucoside moieties and a hydrocarbyl residue, especially an alkyl residue with 8-14 carbon atoms, or 1-2 glucoside moieties and a hydrocarbyl residue, especially an alkyl residue with 8-14 carbon atoms; component I₂ includes hydrocarbyl residues, especially 1-2 alkyl residues, or 1 to 1.5 alkyl residues, each having 8-20 carbon atoms, and a hydrophilic residue bearing more than one, but a maximum of 5, OH groups; said at least one hydrophobic unit of component II₁ is provided at at least one chain end of a water-soluble moiety.

In still another embodiment of the mixture according to the invention, the alkylpolyglucosides of component I₁ have 1-1.5 glucoside moieties and a hydrocarbyl residue, especially an alkyl residue with 8-14 carbon atoms, or 1-2 glucoside moieties and a hydrocarbyl residue, especially an alkyl residue with 8-14 carbon atoms; component I₂ includes hydrocarbyl residues, especially 1-2 alkyl residues, or 1 to 1.5 alkyl residues, each having 8-20 carbon atoms, and a hydrophilic residue bearing more than one, but a maximum of 5, OH groups; further, either the number average molecular weight of each hydrophobic moiety of component II₁ is between 80 and 1000 g/mol, especially between 110 and 500 g/mol, in particular between 110 and 280 g/mol, or the hydrophobic moiety of component II₁ is a hydrocarbyl residue, especially an alkyl residue, which includes from 6 to 50 carbon atoms, preferably from 8 to 20 carbon atoms, or component II is an alcohol ethoxylate consisting of a monovalent alcohol with 8-20 carbon atoms and 25-500 ethylene oxide moieties.

In another embodiment of the mixture according to the invention, component I₁ comprises alkyl glucosides with 6-8 carbon atoms (e.g., hexyl- and octylglucosides) and sulfonates (di-, poly-, alkylaryl sulfonates, such as sodium cumenesulfonate, which exhibit a hydrotopic effect. An additional embodiment of the mixture according to the invention may comprise so-called “builders” (e.g., sodium phosphates, sodium carbonates, sodium silicates, polyphosphates, phosphonic acids, sodium gluconates, borates, polycarboxylates, EDTA etc.).

Builders are complexing agents that bind alkaline earth metals in the emulsion and thus stabilize it.

Another embodiment of the mixture according to the invention may contain so-called “boosters” as foaming agents, which enhance the cleaning effect, and/or wetting agents (e.g., alkyl polyglucosides, phosphonic acids, glycol ethers based on ethylene glycol and propylene glycol moieties, such as diethylene glycol monobutyl ether, and AOT (sodium salt of 1,4-bis(2-ethylhexyl)sulfosuccinate)).

Wetting agents are surfactants that can contribute to an enhancement of the cleaning effect and stabilization of the microemulsion and are not foaming agents.

Both the mixture according to the invention and the emulsion according to the invention can be employed for use in a cleaning agent. In one embodiment, said cleaning agent comprises a microemulsion or bicontinuous microemulsion.

In still another embodiment of the cleaning agent according to the invention, the total surfactant concentration is less than 15%, especially less than 12%, or 9%, or 7%. Depending on the field of application, this very low total surfactant content (content of surface-active agents) enables the preparation of products that are not subject to a labeling obligation with respect to their surfactant content.

The cleaning agent according to the invention is particularly suitable as a replacement for organic solvents. This results in a reduction of the amount of organic solvent employed, up to dispensing with aromatic solvents, which is advantageous in view of working place protection and environmental protection. In addition, both cleaning agents according to the invention and the microemulsions according to the invention contained therein have increased flash points as compared to the organic phases contained therein.

Further, the use of the cleaning agent according to the invention for cleaning off paints, especially partially dried or dry paints, lacquers and tarry compounds and adhesives, as general purpose cleaners and neutral cleaners in the household, in the industry and commercial field is possible.

Using the cleaning agent according to the invention is also recommendable for the cleaning off of paints and lacquers on an aqueous and organic base, especially for cleaning paintbrushes.

The cleaning agent according to the invention may further be used for cleaning off paints, lacquers, oil and/or salt-like residues of metal and/or plastic surfaces.

Such use is recommendable for sensitive surfaces, especially those subject to attack from organic solvents or acidic or alkaline cleaning agents, such as aluminum surfaces. Thus, the cleaning agent according to the invention could replace organic cleaning agents in many fields of application, for example.

The cleaning agent according to the invention may advantageously be used in the printing industry, especially for removing printing inks and paper dust build-up on printing machines and printing formes. It is suitable, for example, for removing printing inks on an aqueous or oil base and of radiation-curable printing ink. Further, the cleaning agent will be applicable in the cleaning of printing cylinders, printing rolls and surfaces of printing machines, preferably for the cleaning of printing machines for conventional printing as well as of printing formes, for example, when the printing process is interrupted, and for non-impact printing methods. The conventional printing methods with printing formes in which the cleaning agent may be employed include planographic printing, gravure printing, letterpress printing, flexographic printing and screen printing; offset printing and waterless offset printing are to be pointed out in particular. The non-impact printing methods without a printing forme include electrophotography, ionography, magnetography, ink jet printing and thermography.

For the stated cleaning purposes, especially in offset printing, cleaning works need to be done on a regular basis in the normal production operation. These are performed either by manual cleaning or by using automated cleaning systems. The cleaning agents employed include organic solvents. Before extended interruptions of the production (e.g., at the weekend), the ink-bearing parts of the machine are cleaned by means of solvents. In addition, printing formes, especially planographic printing formes, must be carefully freed from residual ink when the printing process is interrupted. In addition to the rubber blanket washing systems, modern printing plants are in part also equipped with ink unit washing means. Otherwise, cleaning is performed manually by means of cleaning cloths. Within the scope of maintenance and servicing, the damping systems of the printing plants are also emptied and cleaned on a regular basis.

In manual cleaning, the detergent is applied to rubber blankets with a cleaning cloth. For the ink rolls, application is effected using a spray bottle. The mixture according to the invention contained in the cleaning agent will partially dissolve the ink and can then be removed from the rubber blanket or the ink rolls. In the manual cleaning of the rubber blanket cylinder, the application of the cleaning agent is effected by means of a cleaning cloth to the surface of the rubber blanket. Under a slight pressure, the film containing cleaning agent as well as partially dissolved ink residues and paper components, for example, is washed off with a cleaning cloth. Problems are often caused by residues of color pigments, paper coating, calcium carbonate and other minerals in the pores of the ink roll. They cause the ink rolls and the printing plates to “run blind”. With conventional mixtures of surfactant, such residues cannot be removed.

In offset printing, the ink unit, printing plate, rubber blanket on the rubber cylinder and the impression cylinder are to be cleaned when the order is changed depending on the operational state and requirements. For cleaning the ink unit and the cylinder surfaces, automated washing systems are available that differ in the kind of technical design. In a brush washing means, the cleaning is done by means of a brush roll. Via such a brush roll, the supplied cleaning liquid is transferred to the surface to be cleaned (rubber cylinder, impression cylinder and ink unit). The blanket of the blanket washing means is supplied with cleaning liquid in a finely dosed way by, for example, nozzle strips. The cleaning blanket is pressed against the surface to be cleaned (rubber cylinder, impression cylinder and ink unit).

As to the situation during proof printing and final run in a roll offset operation, the use of aqueous cleaning agents may cause the paper web to break upon contact with the fed-in paper web due to the moisture penetration of the paper printing substrate. This is to be observed, in particular, when used in automated cleaning systems.

With its aqueous fraction, the cleaning agent according to the invention has the advantage that the paper dust is removed along during the cleaning, but without leading to the problem of breaking paper webs as set forth in the previous paragraph.

When printing problems occur, and to ensure a uniform product quality, intermediate cleaning steps are performed with the ink-transferring rubber blankets. Automated cleaning systems are employed for this purpose. About 80% of the heat-set machines in Germany are equipped with automated (rubber blanket) washing systems. Depending on the type of application, 55% work with blanket sheets, 30-35% with brush systems, and 10-15% with spray systems. Otherwise, the cleaning is done manually. Currently, about 90% of the cleaning agents employed in heat-set printing are volatile organic compounds (vapor pressure>0.01 kPa/20° C.), and the remaining 10% are higher boiling cleaning agents based on mineral or vegetable oils or mixtures thereof.

The emulsion according to the invention may further be employed in the food, pharmaceutical or chemical industry.

The invention further relates to a cosmetic article that includes the emulsion according to the invention.

In addition, the emulsion according to the invention is suitable for the preparation of a food, pesticide, especially herbicide, or medicament.

Finally, this invention relates to a process for the preparation of the emulsion according to the invention, wherein components I₁, I₂ and II are mixed. The components of the microemulsion mixtures may be mixed in any order. Preferably, the readily water-soluble components are preliminarily dissolved in water, and the readily oil-soluble components are preliminarily dissolved in oil. Vigorous stirring and optionally heating accelerates the mixing process.

The invention is further illustrated by means of the following Examples.

### EXAMPLES

The drinking water employed is characterized by the following features: pH=8.0; sodium 14 mg/ml; potassium 2.7 mg/ml; calcium 60 mg/ml; magnesium 14 mg/ml; nitrate 34.9 mg/ml; chloride 46.1 mg/ml.

Ketrul D85 (Total) is a mixture of aliphatic hydrocarbons having a flash point of 82° C.

Hydroseal G232H is a mixture of aliphatic hydrocarbons having a flash point of 103° C.

Span 20 (Uniqema): Sorbitan monolaurate, 100% content of active substance.

Imwitor 928 (Sasol): Glyceryl mono-, di- or tricocoate, 100% content of active substance.

Hydropalat 225 (Cognis): Alkylpolyglucoside with C_{8/10} alkyl chain length, 70% content of active substance.

Hydropalat 600 (Cognis): Alkylpolyglucoside with C_{12/14} alkyl chain length, 51.5% content of active substance.

AG 6210 (Akzo Nobel): Alkylpolyglucoside with C_{8/10} alkyl chain length, 60% content of active substance.

1,2-Decanediol (Aldrich): 98% content of active substance.

Brij 700 (Uniqema): PEG-100 stearyl ether, 100% content of active substance.

C12E190 and C12E480 are alcohol ethoxylates consisting of n-dodecanol onto which 190 or 480 ethylene oxide, respectively, have been polymerized.

Sodium gluconate (Dr. Paul Lohmann): sodium gluconate, 100% content of active substance.

DME (Clariant), dipropyleneglycol dimethyl ether, 100% content of active substance.

Zusolat 1004 (Zschimmer & Schwarz): fatty alcohol ethoxylate with 5EO, 85% content of active substance.

The temperature stability of the microemulsions was determined in a thermostatted water bath by visual inspection in transmission. Thus, the mixtures were examined in closed cylinder-shaped glass vessels having diameters of about 5-15 mm, and when the microemulsions exhibited a high turbidity, cuvettes having a layer thickness of 1 mm were used. The temperature phase boundaries of the one-phase region of the microemulsion could be recognized from the drastically increasing turbidity when the stability window was exceeded or fallen short of. Lamellar phases were determined by means of crossed polarizers. In the stability ranges stated for the Examples, there are basically one-phase microemulsions that do not include lamellar phases.

The total surfactant contents relate to the fraction of active substance in the surfactant components and the polymeric additive. All percentages relate to the weight of the ingredients.

### Example 1

Drinking water: 39.81%
Sodium tripolyphosphate: 1.23%
Ketrul D85: 47.52%
Butyldiglycol: 1.90%
Span 20: 5.63%
Hydropalat 225: 3.05%
Brij 700: 0.86%

The range of stability of the microemulsion is between 11 and 28° C., total surfactant content: 8.6%.

### Example 2

Drinking water: 46.45%
Hydroseal G232H: 42.38%
Span 20: 4.88%
AG 6210: 5.39
Brij 700: 0.90%

The range of stability of the microemulsion is between 0 and 52° C., total surfactant content: 9.0%.

### Example 3

Drinking water: 37.60%
Ketrul D85: 49.98%
Imwitor 928: 5.41%
AG 6210: 6.01%
Brij 700: 1.00%

The range of stability of the microemulsion is between 43 and 71° C., total surfactant content: 10.0%.

### Example 4

Drinking water: 38.99%
Ketrul D85: 51.07%
Imwitor 928: 4.33%
AG 6210: 4.81%
Brij 700: 0.80%

The range of stability of the microemulsion is between 44 and 72° C., total surfactant content: 8.0%.

### Example 5

Drinking water: 43.84%
Ketrul D85: 48.41%
Imwitor 928: 3.22%
AG 6210: 3.94%
C12E190: 0.59%

The range of stability of the microemulsion is between 15 and 75° C., total surfactant content: 6.2%.

### Example 6

Drinking water: 43.73%
Ketrul D85: 48.47%
Imwitor 928: 3.24%
AG 6210: 3.97%
C12E480: 0.59%

The range of stability of the microemulsion is between 11 and 70° C., total surfactant content: 6.2%.

### Example 7

Drinking water: 39.71%
Sodium tripolyphosphate: 1.26%
Ketrul D85: 48.85%
Butyldiglycol: 1.94%
Span 20: 2.93%
Hydropalat 600: 4.73%
Brij 700: 0.58%

The range of stability of the microemulsion is between 13 and 42° C., total surfactant content: 5.9%.

### Example 8

Drinking water: 36.06%
Sodium tripolyphosphate: 1.21%
Ketrul D85: 46.59%
Butyldiglycol: 1.86%
Span 20: 4.25%
Hydropalat 600: 9.04%
Brij 700: 0.99%

The range of stability of the microemulsion is between 0 and 26° C., total surfactant content: 9.9%.

### Example 9

Drinking water: 49.89%
Ketrul D85: 37.98%
1,2-Decanediol: 3.43%
AG 6210: 7.80%
Brij 700: 0.90%

The range of stability of the microemulsion is between 13 and 33° C., total surfactant content: 9.0%.

### Example 10

The flash points were measured with the microemulsions from Examples 1 and 7. The flash points determined were 90° C. and 92° C.

The flash point of Ketrul D85 is 82° C.

### Example 11

Drinking water: 31.60%
Sodium gluconate: 2.30%
Dipropylene glycol dimethyl ether: 8.60%
Ketrul D85: 41.70%
Span 20: 7.00%
AG6210: 6.70%
Zusolat 1004: 1.40%
Brij 700: 0.70%

The range of stability of the microemulsion is between 5 and 40° C.; the total surfactant content is 12.9%.

Within the scope of a comparative experiment, the rubber blankets of a rotary offset printing machine with a commercially available oil-based offset printing ink (from Huber) were cleaned on the one hand with a cleaning agent based on an organic solvent (mainly aliphatic hydrocarbons, white petrols) and on the other hand with the microemulsion according to the invention. The cleaning performance, i.e., the removal of the printing ink and of the paper dust build-up, i.e., the solid residues from paper fibers, was essentially the same. After the cleaning, the rolls were cleaner and drier as compared to the case where organic solvents were used as cleaning agents, which resulted in a reduced start-up waste. When the microemulsion was used, the expenditure of work in the manual cleaning of the rubber blankets was lower.

### Example 12

Drinking water: 31.60%
Sodium gluconate: 2.30%
Dipropylene glycol dimethyl ether: 8.60%
Ketrul D85: 41.70%
Span 20: 7.00%
AG6210: 6.70%
Zusolat 1004: 1.40%
Brij 700: 0.70%

The range of stability of the microemulsion is between 5 and 40° C.; the total surfactant content is 12.9%.

Within the scope of a comparative experiment, commercially available paint-brushes contaminated with an acrylate-based paint (white paint from Classic) and an alkyd resin-based paint (color paint from Classic) were cleaned on the one hand with a cleansing spirit (paintbrush cleaner from Classic) and on the other hand with the microemulsion. In both cases, the cleaning performance, i.e., the removal of the paint residues from the paintbrush bristles, was essentially the same. In particular, the residues of the microemulsion could be easily removed by simple rinsing with water. In haptic properties and subsequent renewed wetting, no difference was found.

### REFERENCES

[1] Kahlweit, Strey, Angew. Chem. Int. Ed. Engl. 24, 654 (1985).
[2] Sottmann, Strey, J. Chem. Phys. 106, 8606 (1997).
[3] Stubenrauch, Current Opinion in Colloid & Interfacial Science 6, 160 (2001).
[4] Sottmann et al., Langmuir 18, 3058 (2002).
[5] Aramaki et al., J. Colloid Interface Sci. 196, 74 (1997).
[6] Binks et al., Langmuir 13, 7030 (1997).
[7] Silas, Kaler, J. Colloid Interface Sci. 243, 248 (2001).
[8] Kahlweit, Strey, Angew. Chem. Int. Ed. Engl. 24, 654 (1985).
[9] Sottmann, Strey, J. Chem. Phys. 106, 8606 (1997).
[10] Griffin, W. C., Classification of surface active agents by HLB, J. Soc. Cosmet. Chem. 1, 1949.
[11] Advanced Materials, 2000, 12, Nr. 23, 1751 ff.

1-32. (canceled)

33. An emulsion obtainable by diluting a mixture with an aqueous solution and an oily phase, said mixture comprising:
1.1 80-99% by weight, especially 85-95% by weight, of a component I, based on the total active surfactant content of the emulsion, comprising:
1.1.1 from 80 to 20% by weight of a first surfactant component I₁, which is an alkylpolyglucoside containing 1-2 glucoside moieties and a hydrocarbon moiety, especially an alkyl residue of 6-16 carbon atoms;
1.1.2 from 20 to 80% by weight of a component I₂, which is a second surfactant containing alcohol groups other than an alkylpolyglucoside, the weight proportions being based on component I only; and
1.2 1-20% by weight, especially 5-15% by weight, of a polymeric additive as component II, based on the total active surfactant content of the emulsion, wherein
1.2.1 the polymeric additive, as component II₁, contains at least one water-soluble moiety, especially a linear polymer, preferably having at least a number average molecular weight of at least 500 g/mol, and at least one hydrophobic moiety, the ratio of the number average molecular weights of all water-soluble moieties to the number average molecular weights of all hydrophobic moieties being from 2:1 to 1000:1, especially from 5:1 to 200:1, in particular from 10:1 to 50:1, wherein each of said at least one hydrophobic moieties has a number average molecular weight of at most 1000 g/mol, especially from 80 to 1000 g/mol, especially from 110 to 500 g/mol, in particular from 110 to 280 g/mol; or
1.2.2 the polymeric additive, as component II₂, contains at least one water-soluble moiety and at least one hydrophobic moiety and is an amphiphilic comb polymer including a backbone with two or more side chains attached to said backbone, wherein the side chains are distinguished from one another and/or from the backbone in terms of their amphiphilic character; or
1.2.3 the polymeric additive, as component II₃, contains at least one water-soluble moiety and at least one hydrophobic moiety wherein component II₃ is an ABA or BAB triblock copolymer with water-soluble A blocks and hydrophobic B blocks; and
1.3 optionally further surfactants.

34. The emulsion according to claim 33, wherein component I₂ comprises hydrocarbons, especially 1-2 alkyl residues, preferably 1 to 1.5 alkyl residues, each having 8-20 carbon atoms, and a hydrophilic residue bearing more than one, but a maximum of 5, OH groups.

35. The emulsion according to claim 33, wherein the alkylpolyglucosides of component I₁ have 1-1.5 glucoside moieties and a hydrocarbyl residue, especially an alkyl residue with 8-14 carbon atoms, or 1-2 glucoside moieties and a hydrocarbyl residue, especially an alkyl residue with 8-14 carbon atoms.

36. The emulsion according to claim 34, wherein the hydrocarbyl residues, especially the alkyl residues, of component I₂ are connected with the hydrophilic residue via ether or ester groups.

37. The emulsion according to claim 34, wherein the hydrocarbyl residues, especially the alkyl residues, of component I₂ are connected with the hydrophilic residue via carbon bonds.

38. The emulsion according to claim 34, wherein component I₂ comprises a hydrocarbyl residue, especially an alkyl residue with 10-18 carbon atoms, preferably 10-14 carbon atoms.

39. The emulsion according to claim 34, wherein the hydrophilic residue of component I₂ comprises 1.5-3 OH groups.

40. The emulsion according to claim 34, wherein the hydrophilic residue of component I₂ is not ethoxylated.

41. The emulsion according to claim 33, wherein component I₂ is a sorbitan ester, a polysorbate, a glycerol monoester, a mixture of glycerol monoester and glycerol diester, a monoester or diester of pentaerythritol, a monoether or diether of pentaerythritol, 1,2-decanediol or 1,2-dodecanediol.

42. The emulsion according to claim 33, wherein said at least one hydrophobic moiety of component Hᵢ is provided at at least one chain end of a water-soluble moiety.

43. The emulsion according to claim 33, wherein the number average molecular weight of the water-soluble A blocks and the hydrophobic B blocks of the diblock copolymer or a triblock copolymer of component II₃ according to claim 1 is between 500 and 100,000 g/mol, especially between 2000 and 20,000 g/mol and especially between 3000 and 10,000 g/mol.

44. The emulsion according to claim 33, wherein the water-soluble moiety of component II₁ is not ionic or ionic in nature or is constituted of an ionic and a non-ionic component.

45. The emulsion according to claim 33, wherein the hydrophobic moiety of component II₁ is a hydrocarbyl residue, especially an alkyl residue, especially having from 6 to 50 carbon atoms, preferably from 8 to 20 carbon atoms.

46. The emulsion according to claim 33, wherein component II₁ is an alcohol ethoxylate consisting of a monovalent alcohol with 8-20 carbon atoms and 25-500 ethylene oxide moieties.

47. The emulsion according to claim 33, characterized by being a microemulsion, in particular being a bicontinuous microemulsion.

48. A cleaning agent comprising the emulsion according to claim 33, especially having a total surfactant concentration of less than 15%, especially less than 12%, or 9%, or 7%.

49. Use of the cleaning agent according to claim 48 in the printing industry, especially in offset printing, especially for removing printing inks and paper dust build-up on printing machines and printing formes, for the cleaning of printing cylinders, printing rolls and surfaces of printing machines, preferably for the cleaning of printing machines for conventional printing as well as of printing formes when the printing process is interrupted, for cleaning off paints, lacquers, paper dust build-up, salty, oily and tarry compounds and adhesives, as general purpose cleaners and neutral cleaners in the household, in the industry and commercial field, for cleaning off paints and lacquers on an aqueous and organic base, especially for cleaning paintbrushes, for cleaning off paints, lacquers, oil and/or salty residues from metallic and/or plastic surfaces.

50. A cosmetic article of foodstuff comprising the emulsion according to claim 33.
